SEQUENCE LISTING (3 sequences)

SEQ ID NO: 1 - DNA, 42 bp, Homo sapiens
tgcacctttgtttgaacccacatcttctgcaaagaacaccaa

SEQ ID NO: 2 - protein, 14 aa, Homo sapiens
LVFFAEDVGSNKGA

SEQ ID NO: 3 - DNA, 37 bp, Homo sapiens
tgcacctttgtttgaacccacatcttctgcaaagaac

(19) United States
(12) Patent Application Publication
(10) Pub. No.: US 2011/0166197 A1
(43) Pub. Date: Jul. 7, 2011

(54) Antisense Modulation Of Amyloid Beta Protein Expression

(75) Inventors: Thomas Darling, Saint Louis, MO (US); Vijaya B. Kumar, Saint Louis, MO (US); William A. Banks, Saint Louis, MO (US); Susan Farr, Saint Louis, MO (US); John E. Morley, Saint Louis, MO (US)

(73) Assignee: EDUNN BIOTECHNOLOGY, INC., St. Louis, MO (US)

(21) Appl. No.: 12/918,402

(22) PCT Filed: Feb. 19, 2009
(86) PCT No.: PCT/US2009/034561

§ 371 Date: Mar. 9, 2011

Related U.S. Application Data
(60) Provisional application No. 61/029,907, filed on Feb. 19, 2008.

(51) Int. Cl. A61K 31/7088; C07H 21/04; C12N 5/071; A61P 25/28; A61P 25/16; A61P 25/00; A61P 7/04

(52) U.S. Cl. 514/44 A; 536/23.1; 536/24.5; 514/44 R; 435/375

(57) ABSTRACT

Antisense nucleic acids, compositions and methods are provided for modulating the expression of an amyloid beta protein (AβP) portion of the amyloid precursor protein (APP) coding sequence. The compositions comprise antisense nucleic acids targeted to nucleic acids encoding amyloid precursor protein. Methods of using these nucleic acids for modulation of amyloid precursor protein expression and for treatment of diseases and conditions associated with expression of the amyloid beta protein portion of the amyloid precursor protein are provided.

This application claims priority to U.S. provisional application No. 61/029,907, filed Feb. 19, 2008, the disclosure of which, along with all documents cited therein, is incorporated by reference in its entirety.

Compositions and methods for modulating the expression of the human amyloid precursor protein are described herein. In particular, this invention relates to antisense compounds, particularly oligonucleotide compounds, which, in preferred embodiments, hybridize with nucleic acid molecules involved in the synthesis of amyloid precursor protein (APP), particularly messenger ribonucleic acids (mRNAs) or deoxyribonucleic acids (DNAs) encoding the amyloid beta protein (AβP) portion of APP. Such compounds are shown herein to modulate the expression of the AβP portion of APP.

The compounds find particular use in the treatment of patients having diseases where accumulation of neuritic plaques are prevalent. The disease states or maladies include but are not limited to Alzheimer's disease, familial Alzheimer's disease, Down's Syndrome and homozygotes for the apolipoprotein E4 allele.

Alzheimer's disease (AD), first described by the Bavarian psychiatrist Alois Alzheimer in 1907, is a progressive neurological disorder that begins with short-term memory loss and is characterized by a progressive decline in cognitive function and behavior. Progression of the disease leads to disorientation, impairment of judgment, reasoning, attention and speech and, ultimately, dementia. The course of the disease usually leads to death in a severely debilitated, immobile state between four and 12 years after onset. AD has been estimated to afflict 5 to 11 percent of the population over age 65 and as much as 47 percent of the population over age 85. The societal cost for managing AD is upwards of $100 billion annually, primarily due to the extensive custodial care required for AD patients. Moreover, as adults born during the population boom of the 1940's and 1950's approach the age when AD becomes more prevalent, the control and treatment of AD will become an even more significant health care problem. Despite continuous efforts aimed at understanding the physiopathology of AD, there is currently no treatment that significantly retards the progression of the disease.

Pathologically, Alzheimer's disease is a neurodegenerative disorder characterized by the presence of extracellular senile plaques and intracellular neurofibrillary tangles in the brains of affected individuals. (Masters, C. L. et al., Proc. Natl. Acad. Sci. USA, 82:4245-4249 (1985)). The senile plaques, found in abundance in Alzheimer's disease-affected brain cells, are composed of a core of extracellular AβP (also referred to in the art as beta-amyloid protein or A4 protein) surrounded by reactive cells and degenerating neurites. (Lenders, M. B. et al., Acta Neurologica Belgica, 89:279-285 (1989); and Perry, G. et al., Lancet, 2:746 (1988)). While the plaques form primarily in particular parts of the brain—such as the hippocampus—in some cases they are also found in the walls of cerebral and meningeal blood vessels. (Delacourt, A. et al., Virchows Archiv.—A, Pathological Analomy & Histopathology, 411:199-204 (1987); and Masters, C. L. et al., EMBO Journal, 4:2757-2763 (1985)). The presence of these plaques is the essential observation underpinning the amyloid hypothesis that the prevention of excessive amyloid biosynthesis or metabolism, including aggregation that can lead in late stage disease to amyloid plaque formation, will prevent the onset of Alzheimer's disease. Formation of amyloid plaques and Alzheimer's disease-related manifestations is also a frequent complication of Down's Syndrome patients in middle age. While most cases of Alzheimer's disease are “spontaneous,” meaning that there is no familial history of the disease and hence no known genetic linkage that would predispose a person to developing Alzheimer's disease, early-onset familiar forms of Alzheimer's disease have also been observed in which mutations in the AβP portion of the APP gene have been observed which accelerate amyloid production. (Dingwall, J. Clin. Invest.; 2001, 108, 1243-1246; Kienlen-Campard et al., Exp. Gerontol., 2000, 35, 843-850).

The major protein subunit of the senile plaques, AβP is a 4 kD (39-43 amino acid) protein that is a cleavage product of the much larger APP. Whereas APP is a transmembrane protein with no known harmful physiological effects, AβP is known to be highly aggregating and to deposit and form plaques and to accumulate at high levels in the brain in Alzheimer's disease, Down's Syndrome, related diseases, and some normal aged individuals. (Verga, L. et al., Neuroscience Letters, 105:294-299 (1989)). Strong evidence that AβP deposition plays a critical role in the development of Alzheimer's disease came from the identification of familial Alzheimer's disease kindreds in which the Alzheimer's disease phenotype co-segregates with mutations from the APP gene. (Younkin, S. G., Tohuku J. of Exper. Med., 174:217-223 (1994); and Matsumura, Y. et al., Neurology, 46:1721-1723 (1996)).

Nucleic acid sequences for APP, AβP, and related proteins have been reported by Ponte et al., (U.S. Pat. No. 5,220,013), and Greenberg et al., (WO88/03951), among others. Amyloid precursor protein has several isoforms generated by alternative splicing of a 19-exon gene made up of exons 1-13, 13a, and 14-18 (Yoshikai et al., Gene, 87:257 (1990)). The predominant transcripts are APP695 (exons 1-6, 9-18, not 13a); APP751 (exons 1-7, 9-18, not 13a); and APP770 (exons 1-18, not 13a). All of these encode multidomain proteins with a single membrane spanning region. The AβP segment of APP comprises approximately one-half of the transmembrane domain and approximately the first 28 amino acids of the extracellular domain of an APP isoform. (U.S. Pat. No. 5,455,169). In this structure, the 42 amino acid sequence of the AβP segment of human APP is often shown having its C-terminal to the left and an N-terminal portion to the right.

The amyloid precursor protein isoforms differ in that APP751 and APP770, but not APP 695, contain exon 7, which encodes a serine protease inhibitor domain. APP695 is a predominant form in neuronal tissue, whereas APP751 is the predominant variant elsewhere. AβP is derived from that part of APP encoded by parts of exons 16 and 17.

Two major pathways of APP processing in vivo have been described. Normal processing of APP in the secretory pathway occurs by proteolytic cleavage within the AβP sequence of the APP resulting in the generation of a large (approximately 100 kD) soluble, secreted N-terminal fragment of the protein (Oltersdorf, T., Nature, 14.341, 144-147 (1989); and de Sauvage, F., and J. N. Octave, Science, 11:245, 651-653 (1989)) and a smaller (approximately 9-10 kD), membrane-associated C-terminal fragment (Wolf, D. et al., EMBO Journal, 9:2079-2084 (1990); and Ghiso, J. et al., Biochemical Journal, 288:1053-1059 (1992). This type of cleavage has been described as occurring at, or near, the position identified as “α-secretase”. Neither of the two protein fragments that result from the cleavage is amyloidogenic (i.e., tends to form senile plaques), because neither of them contains the entire AβP protein.

However, another pathway of APP metabolism involves the endosomal-lysosomal system and results in generation of an amyloidogenic C-terminal fragment of APP. When APP is processed by the endosomal-lysosomal system, a complex set of—COOH terminal derivatives of APP is produced that includes potentially amyloidogenic forms having the entire AβP at, or near, their N-terminal. One form of this aberrant cleavage of APP occurs at, or near, the positions identified as “β and γ secretases” (Glenner and Wong, Biochem. Biophys. Res. Commun., 122:1131-1135 (1984); Volloch, FEBS Letters, 390:124-128 (1996)) and results in the generation of AβP that is known to deposit and form plaques. The plaques have been shown to be associated with the clinical severity of Alzheimer's disease. Abundant deposition of AβP in the brains of patients with Alzheimer's disease has suggested that regulation of APP expression and metabolism are key pathological events. It is known that some amount of AβP is constantly produced in the brain, but is continuously cleared. Apparently, the two alternative pathways of APP metabolism must be precisely balanced in order to avoid the accumulation of AβP in harmful concentrations.

It is known that the APP gene in humans is located on chromosome 21. People with trisomy twenty-one (Down's Syndrome, or DS) possess an extra chromosome that contains the gene that encodes the APP and thus have elevated beta-amyloid levels and invariably develop Alzheimer's disease later in life. Several different studies have suggested the apparent involvement of several particular sites in the APP gene in Alzheimer's disease. Three separate mutations in codon 717 of the APP transcript have been found in familial Alzheimer's disease: val717-to-ile, val717-to-phe, and val717-to-gly. See, Hardy et al., U.S. Pat. No. 5,877,015. The location of these mutations and of the double mutation disclosed by Mullan (U.S. Pat. No. 5,455,169) suggested to Suzuki et al., Science, 264:1336-1340 (1994), that they may cause Alzheimer's disease by altering AβP processing in a way that is amyloidogenic. They found that the APP717 mutations were consistently associated with a 1.5- to 1.9-fold increase in the percentage of longer peptide fragments generated and that the longer peptide fragments formed insoluble amyloid fibrils more rapidly than did the shorter ones. Alternative splicing of transcripts from the single APP gene results in at least 10 isoforms of the gene product (Sandbrink et al., J. Biol. Chem., 269: 1510-1517 (1994)), of which APP695 is preferentially expressed in neuronal tissues. In 3 mutations, valine-642 in the transmembrane domain of APP695 is replaced by isoleucine, phenylalanine, or glycine, in association with dominantly inherited familial Alzheimer's disease. According to an earlier numbering system, val642 was numbered 717 and the 3 mutations were V7171, V717F, and V717G, respectively). Yamatsuji et al., Embo J. 15: 498-509 (1996), stated that these 3 mutations account for most, if not all, of the chromosome 21-linked Alzheimer's disease. In transgenic mice, overexpression of such mutants mimics the neuropathology of Alzheimer's disease. Yamatsuji et al., Science, 272: 1349-1352 (1996), demonstrated that expression of any 1 of these 3 mutant proteins, but not of normal APP695, induced nucleosomal DNA fragmentation in cultured neuronal cells. Induction of DNA fragmentation required the cytoplasmic domain of the mutants and appeared to be mediated by heterotrimeric guanosine triphosphate-binding proteins (G-proteins).

The use of complimentary sequences to arrest translation of mRNAs was described in the late 1970's (See, e.g., Paterson et al., Proc. Natl. Acad. Sci., 74:4370-4374 (1977); Hastie, N. D. and W. A. Held, Proc Natl. Acad. Sci., 75: 1217-1221 (1978); and Zamecnik, P. C. and M. L. Stephenson, Proc. Natl. Acad. Sci., 75:280-284 (1978)). However, the use of antisense oligonucleotides for selective blockage of specific mRNAs is of recent origin. (See, e.g., Weintraub et al, Trends Gen., 1:22-25 (1985); Loke et al., Prod. Natl. Acad. Sci, USA, 86:3474-3478 (1989); Mulligan et al., J. Med. Chem., 36:1923-1937 (1993); and Wagner, Nature, 372:333-335 (1994)). The mechanism of antisense inhibition in cells was previously analyzed and the decrease in mRNA levels mediated by oligonucleotides was shown to be responsible for the decreased expression of several proteins. (See, Walder, R. Y. and J. A. Walder, Proc. Natl. Acad. Sci. USA, 85:5011-5015 (1988); Dolnick, B. J., Cancer Invest., 9:185-194 (199 1); Crooke S, and B. LeBleu, Antisense Research and Applications, CRC Press, Inc., Boca Raton, Fla. (1993); Chiang et al., J. Biol. Chem., 266:18162-18171 (1991); and Bennett et al., J. Immunol., 152:3530-3540 (1994)).

The use of antisense oligonucleotides is recognized as a viable option for the treatment of diseases in animals and man. For example, see U.S. Pat. Nos. 5,098,890, 5,135,917, 5,087,617, 5,166,617, 5,166,195, 5,004,810, 5,194,428, 4,806,463, 5,286,717, 5,276,019, 5,264,423, 4,689,320, 4,999,421 and 5,242,906, which teach the use of antisense oligonucleotides in a variety of diseases including cancer, HIV, herpes simplex virus, influenza virus, HTLV-HIV replication, prevention of replication of foreign nucleic acids in cells, antiviral agents specific to CMV, and treatment of latent EBV infections.

Recently, it has been recognized that regulation of the expression of the APP gene could be useful for the detection and treatment of diseases associated with deposition of the AβP portion. For example, Salbaum et al. (U.S. Pat. No. 5,853,985) reported the use of the promoter for human APP in a method for screening for a drug that regulates the expression of the APP gene. Monia et al., (U.S. Pat. No. 5,837,449) described oligonucleotide probes that could selectively hybridize to an APP gene having mutations at codons 717, 670 and 671 of the APP770 isoform, and serve for detection as well as for modulation of the expression of APP. Besides the mutations at codons 717, 670 and 671 of APP770, Monia et al. suggested that the same mutations at codons 642, 595 and 596 of the shorter isoform—APP695—may be expected to provide similar effects. Nevertheless, the use of such oligonucleotides has not yet been proven to be an effective treatment for diseases involving the expression of AβP.

Currently, there are no known therapeutic agents that effectively inhibit the synthesis of AβP precursor, and to date, investigative strategies aimed at modulating AβP precursor levels have involved the use of transgenic and knockout mice, as well as antisense oligonucleotides, ribozymes, and vectors expressing antisense AβP precursor.

Several antisense oligonucleotides directed against human AβP precursor with GenBank accession number NM_{—}000484 have been reported in the art, including an oligonucleotide targeted to positions 138 to 157 and phosphorothioated at the 5′ and 3′ ends (Hoffmann et al., Eur J. Cell Biol., 2000, 79, 905-914), a phosphorothioated oligonucleotide targeted to positions 148 to 167 (Chang et al., J. Mol. Neurosci., 1999, 12, 69-74), three phosphorothioated and three normal oligonucleotides 20 nucleotides in length and targeted to the AUG codon, the Kunitz protease inhibitory (KPI) domain, and the 13/A4 domain (Suh et al., Ann. N.Y. Acad. Sci., 1996, 786, 169-183), and a phosphorotioated oligonucleotide targeted to positions 138 to 160 (Majocha et al., Cell. Mol. Neurobiol., 1994, 14, 425-437). Claimed in U.S. Pat. No. 6,310,048 and PCT publication WO 01/42266 is an antisense compound comprising a nucleic acid sequence complementary to a nucleic acid sequence that encodes the amino acids of an APP and which inhibits the expression of the beta-amyloid portion of the APP which permitting the expression of a portion of the APP (Kumar 2001).

An impediment to treatment with many neurological therapeutic agents is the human body's blood brain barrier. The blood-brain barrier is the specialized system of microvascular endothelial cells that shields the brain from toxic substances in the blood, supplies brain tissues with nutrients, and filters harmful compounds from the brain back to the bloodstream, all to maintain a homeostatic environment in the central nervous system (Persidsky et al., J. Neuroimmune Pharmacol., 2006, 1, 223-236).

Transport across the blood brain barrier is strictly limited through both physical and metabolic barriers. Unlike the rest of the body outside the brain, where the walls of the capillaries are made up of fenestrated endothelial cells through which soluble chemicals can pass from blood to tissues or vice versa, the capillaries that supply the blood to the brain have tight junctions which block passage of most molecules through the capillary endothelial membranes. While these membranes allow passage of lipid soluble materials, such as oxygen, carbon dioxide, ethanol, and steroid hormones, water-soluble materials such as glucose, proteins, and amino acids do not pass through the blood-brain barrier. Molecules that are charge bearing, large, or hydrophilic require gated control channels, ATP proteins, and/or receptors to facilitate passage through the blood brain barrier (Roney et al., J. of Controlled Release, 2005, 208, 193-214).

As a result of restricted permeability, the blood brain barrier is a limiting factor for the delivery of therapeutic agents into the central nervous system (Persidsky et al., J Neuroimmune Pharmacol., 2006, 1, 223-236). Poor blood brain barrier penetration is a significant problem for drugs targeting diseases of the brain, such as Alzheimer's disease, and affects approximated 98% of the small molecules and nearly 100% of large molecules that are potential therapeutic agents (de Boer, A. G. and Gaillard, P. J., Annu. Rev. Pharmacol. Toxicol., 2007, 47, 323-55). Overcoming the difficulty of delivering therapeutic agents to specific regions of the brain presents a major challenge to treatment of most brain disorders.

Thus, despite significant advances in the understanding of the pathology of Alzheimer's disease and related diseases, there still is a need for methods to regulate the expression of AβP; especially a method that could improve the acquisition and retention capabilities of humans and animals that are affected by, or at risk of being affected by diseases that were related to the deposition of AβP in the brain. There is also a need for such a composition that is capable of crossing the blood brain barrier in an amount sufficient to act therapeutically within the brain.

### SUMMARY OF THE INVENTION

The invention is based, in part, on the discovery of an oligonucleotide that crosses the blood-brain barrier into the central nervous system. Accordingly, in one embodiment, the invention comprises a nucleic acid that has a sequence that is at least 90% identical to SEQ ID NO: 1. In another embodiment, the invention comprises a composition comprising a nucleic acid of the invention and a pharmaceutically acceptable excipient, carrier, or diluent. The nucleic acids of the invention are capable of crossing the blood-brain barrier and may be administered via oral, parenteral, subcutaneous, intraperitoneal, intradermal, intramuscular, intravenous, intraarticular, intracranial, intrathecal, intramedullary, intraventricular, intranasal, pulmonary, by inhalation, by insufflation, transmucosal, transdermal, rectal, topical, dermal, buccal, sublingual or intraocular administration. In one embodiment, they are administered intravenously, subcutaneously, orally, intranasally, or intramuscularly.

In yet another embodiment, the invention comprises a method of inhibiting the expression of amyloid precursor protein in cells or tissues. The method comprises administering a nucleic acid or composition of the invention to the cells or tissues so that expression of APP is inhibited. In still another embodiment, the invention comprises a method of modulating the expression of amyloid beta protein in cells or tissues. The method comprises administering a nucleic acid or composition of the invention to the cells or tissues so that expression of the amyloid beta protein is modulated.

In a further embodiment, the invention comprises a method of treating a subject having a disease or condition associated with amyloid beta protein precursor or amyloid beta protein. The method comprises administering a therapeutically or prophylactically effective amount of a nucleic acid of the invention to the subject so that expression of APP is inhibited. The invention also comprises a method of prophylactic treatment of a patient not previously diagnosed with AD, MCI, dementia, or pre-dementia, which patient displays an elevated level of AβP in the brain. The method comprises administering to the patient an amount of a nucleic acid of the invention effective to prevent or delay development of AD, MCI, dementia, or pre-dementia. The invention also comprises a method of enhancing cognitive function. The method comprises administering to a subject an amount of a nucleic acid of the invention effective to enhance cognitive function.

In its neuroprotective role, the blood-brain barrier functions to hinder the delivery of many potentially important diagnostic and therapeutic agents to the brain. Therefore, it is surprising that the nucleic acids of the invention cross the blood-brain barrier into the central nervous system. The nucleic acids of the invention are therefore capable of therapeutically acting upon cells and tissues within the brain without the need for direct delivery techniques. Direct techniques include intra-cerebral-ventricular or intrathecal infusion or injection of the drug into the cerebral spinal fluid, direct infusion into the brain, convection-enhanced drug delivery, or brain implants. However, even direct techniques are not completely or consistently able to effect global penetration of drugs into the brain (de Boer, A. G. and Gaillard, P. J., Annu. Rev. Pharmacol. Toxicol., 2007, 47, 323-55). The methods, nucleic acids and compositions of the present invention therefore provide for delivery to the central nervous system of compounds that are necessary for treatment modalities in any condition affecting the central nervous system where the blood-brain barrier would impede the delivery of the compound. The methods, nucleic acids and compositions of the present invention are an improvement over currently available means of delivery of compounds to the central nervous system through the blood-brain barrier.

It follows naturally, that another patient group in which the oligonucleotides disclosed herein could find use is patients with Down Syndrome (DS), who begin to succumb to Alzheimer's disease at about age 40 years. It is possible that the DS patients will ultimately be treated with OL-1 h in one of its embodiments, which has consequences for drug design.

The nucleic acids, compositions, and methods disclosed herein could also benefit patients with early diagnosis of AD, or pre-Alzheimer's conditions. During the past several years, scientists have focused on a type of memory change called mild cognitive impairment (MCI), which is different from both AD and normal age-related memory change. People with MCI have ongoing memory problems, but they do not have other losses such as confusion, attention problems, and difficulty with language. However, MCI progresses to AD, and to date no intervention has changed the outcome. The National Institute of Aging (NIA)—funded Memory Impairment Study compared donepezil (Aricept), vitamin E, or placebo in participants with MCI to see whether the drugs might delay or prevent progression to AD. The study found that the group with MCI, taking the drug donepezil, were at reduced risk of progressing to AD for the first 18 months of a 3-year study when compared with their counterparts on placebo. The reduced risk of progressing from MCI to a diagnosis of AD among participants on donepezil disappeared after 18 months, and by the end of the study, the probability of progressing to AD was the same in the two groups. Vitamin E had no effect at any time point in the study when compared with placebo.

Similar conclusions about treatment of MCI patients with cholinesterase inhibitors that are currently prescribed to treat mild-to-moderate AD, which are often prescribed on a so-called “off-label” basis to people with pre-dementia. Though doctors are divided over their effectiveness, due to results such as those from six clinical trials in which in none of these drugs significantly reduced the rate of progression from MCI to dementia, some experts and patient groups have called for such anti-cholinesterase drugs to be given to people with mild cognitive impairment MCI. An unmet medical need clearly exists for efficacious drugs to treat MCI and other pre-Alzheimer's conditions.

Patients with MCI may therefore benefit from treatment with the compounds and compositions disclosed herein. In certain embodiments, the compound may be OL-1 or one or more molecules of related sequence. The inevitable progression could be halted sufficiently early to protect normal learning and memory, by preventing further damage to brain cells.

### BRIEF DESCRIPTION OF THE DRAWINGS

FIG. 1 depicts a flow chart description of the manufacture of OL-1 h, referenced in Example 1.

FIG. 2 shows the effects of OL-1 in a murine model of Down's Syndrome.

FIG. 3 shows the effects of delivery of human OL-1 h into mice by intravenous (IV) administration, and concentration in brain versus serum.

FIG. 4 shows the effects of delivery of murine OL-1 h into mice by subcutaneous (subQ) administration, and concentration in brain versus serum.

FIG. 5 shows the concentrations of radiolabeled oligonucleotides in the serum (FIG. 5a) or the brain (FIG. 5b) after oral administration.

FIG. 6 demonstrates that intranasal administration of OL-1m in mice results in entry into three brain regions.

FIG. 7 shows that intranasal administration of OL-1m in the SAMP8 mouse is efficacious in the T-maze footshock avoidance test.

FIG. 8a compares the level of OL-1 h in mouse blood after intranasal or IV administration at various time points.

FIG. 8b compares the level of OL-1 h in mouse brain after intranasal or IV administration at various time points.

### DETAILED DESCRIPTION

Described herein are antisense compounds, especially nucleic acid and nucleic acid-like oligomers, which are targeted to nucleic acids encoding the amyloid beta protein (AβP) portion of amyloid precursor protein (APP), and which modulate the expression of the AβP portion of APP. Certain oligonucleotides of the invention are designed to bind either directly the mRNA transcribed from, or to a selected DNA portion of, the APP, thereby modulating the amount of AβP translated from an APP mRNA or the amount of mRNA transcribed from an APP gene, respectively.

One embodiment of the present invention is an antisense compound 42 nucleobases in length targeted to a nucleic acid molecule encoding a human APP which modulates the expression of human APP. Preferably, the antisense compound is an antisense oligonucleotide. In this embodiment, the present invention is directed to novel oligonucleotides, in particular the sequence TGC ACC TTT GTT TGA ACC CAC ATC TTC TGC AAA GAA CAC CAA (SEQ ID NO: 1), which is complementary to nucleotides that encode for amino acids 17-30, Leu Val Phe Phe Ala Glu Asp Val Gly Ser Asn Lys Gly Ala (SEQ. ID NO: 2) of the AβP portion of APP. In certain embodiments, the antisense oligonucleotide is complementary to nucleotides that encode for amino acids 17-30 (SEQ. ID NO: 2) of the AβP portion of human APP. In certain embodiments, the antisense oligonucleotide comprises at least one modified internucleoside linkage.

Pharmaceutical and other compositions comprising these compounds are also provided. Methods of treating an animal, particularly a human, suspected of having or being prone to a disease or condition associated with production of an abnormal amount of the AβP portion of APP are also set forth herein. Such methods comprise administering a therapeutically or prophylactically effective amount of one or more compounds or compositions to the person in need of treatment. Another embodiment of the present invention is a method of inhibiting the expression of APP in human cells or tissues comprising contacting the cells or tissues with the antisense compound described above so that expression of APP is inhibited.

Another embodiment of the present invention also provides a pharmaceutical composition comprising the antisense compound described above and a pharmaceutically acceptable carrier or diluent. Preferably, the antisense compound is an antisense oligonucleotide. In one aspect of this preferred embodiment, the antisense compound is targeted to the portion of the APP that is metabolized through the endosomal-lysosomal system, resulting in potentially amyloidogenic forms resulting from aberrant cleavage of APP at, or near, the positions identified as “β and γ secretases.” The antisense compound preferentially inhibits the expression of APP.

A need exists for new technologies to address the expression of APP, targeted specifically towards modulating the production of the amyloidogenic AβP portion of the APP produced by the cleavage of APP at or near the β and γ secretase cleavage positions. Antisense oligonucleotide technology is an effective means for reducing the expression of specific gene products and is therefore useful in a number of therapeutic, diagnostic, and research applications for the modulation of APP expression.

Described herein are compositions and methods for modulating AβP expression by modulating the expression of the APP, one of whose cleavage products is APP. This is accomplished by providing oligonucleotides which specifically hybridize with one or more nucleic acid molecules encoding APP. In certain embodiments, single oligonucleotides may be used to target APP.

The present invention employs oligonucleotides for use in antisense inhibition of the function of RNA and DNA encoding APP protein. The present invention also employs oligonucleotides which are designed to be specifically hybridizable to DNA or mRNA encoding such proteins and ultimately to modulate the amount of such proteins transcribed from their respective genes. Such hybridization with mRNA interferes with the normal role of mRNA and causes a modulation of its function in cells. The functions of mRNA to be interfered with include all vital functions such as translocation of the RNA to the site for protein translation, actual translation of protein from the RNA, splicing of the RNA to yield one or more mRNA species, and catalytic activity or complex formation which may be engaged in or facilitated by the RNA. The overall effect of such interference with mRNA function is modulation of the expression of either a portion or the entirety of an APP protein, wherein “modulation” means either an increase (stimulation) or a decrease (inhibition) in the expression of a portion or the entirety of an APP protein. In the context of the present invention, inhibition is the preferred form of modulation of gene expression and mRNA is often a preferred target nucleic acid.

The antisense oligonucleotides of the invention are useful in treating diseases or conditions such as, but not limited to, any variant of Alzheimer's disease, mild cognitive dysfunction, Down's Syndrome, Parkinson's disease, familial Alzheimer's disease, homozygosity for the apolipoprotein E4 allele, Dementia pugilistica (including head trauma), Hereditary Cerebral Hemorrhage with amyloidosis of the Dutch type (HCHWA-D), vascular dementia with amyloid angiopathy, MCI, dementia, pre-dementia, or any other disease states arising from inappropriate or over-expression of the AβP protein produced by cleavage of APP at or near the β and γ secretase cleavage positions.

As used herein, the following terms have the meanings indicated.

As used herein, the terms “target nucleic acid” and “nucleic acid molecule encoding APP” have been used for convenience to encompass DNA encoding at least a portion of APP, RNA (including pre-mRNA and mRNA or portions thereof) transcribed from such DNA, and also cDNA derived from such RNA. The hybridization of a compound with its target nucleic acid is generally referred to as “antisense”. Consequently, the preferred mechanism believed to be included in the practice of some embodiments is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

As used herein, antisense compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds may elicit the action of one or more enzymes or structural proteins to effect modification of the target nucleic acid.

The antisense compounds also include modified compounds in which a different base is present at one or more of the nucleotide positions in the compound. For example, if the first nucleotide is an adenine, modified compounds may be produced which contain thymine, guanine or cytosine at this position. This may be done at any of the positions of the antisense compound. These compounds are then tested using the methods described herein to determine their ability to inhibit expression of APP mRNA.

As used herein, the term “oligomeric compound” refers to a polymer or oligomer comprising a plurality of monomeric units. While oligonucleotides are a preferred form of antisense compound, other families of antisense compounds are contemplated as well, including but not limited to oligonucleotide analogs and mimetics such as those described herein.

In one preferred embodiment, the antisense compounds are 30 to 50 nucleobases in length. One having ordinary skill in the art will appreciate that this embodies compounds of 30, 31, 32, 33, 34, 35, 36, 37, 38, 39, 40, 41, 42, 43, 44, 45, 46, 47, 48, 49, or 50 nucleobases in length.

Particularly preferred compounds are oligonucleotides from about 40 to about 45 nucleobases, even more preferably those comprising about 42 nucleobases.

Antisense compounds 8 to 80 nucleobases in length comprising a stretch of at least eight (8) consecutive nucleobases selected from within the illustrative antisense compounds are considered to be suitable antisense compounds as well.

One having skill in the art armed with the preferred antisense compounds illustrated herein will be able, without undue experimentation, to identify further preferred antisense compounds.

As used herein, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

The oligonucleotides in accordance with this invention preferably comprise from about 30 to about 50 nucleotides. It is more preferred that such oligonucleotides comprise from about 40 to 45 nucleotides. As is known in the art, a nucleotide is a base-sugar combination suitably bound to an adjacent nucleotide through a phosphodiester, phosphorothioate or other covalent linkage.

The oligonucleotides used in accordance with this invention may be conveniently and routinely made through the well-known technique of solid phase synthesis. Equipment for such synthesis is sold by several vendors including, for example, Agilent Technologies (Santa Clara, Calif.). Any other means for such synthesis known in the art may additionally or alternatively be employed. It is also known to use similar techniques to prepare other oligonucleotides such as the phosphorothioates and alkylated derivatives.

Oligonucleotides may comprise nucleotide sequences sufficient in identity and number to effect specific hybridization with a particular nucleic acid. Such oligonucleotides which specifically hybridize to a portion of the sense strand of a gene are commonly described as “antisense oligonucleotides.” Antisense oligonucleotides are commonly used as research reagents, diagnostic aids, and therapeutic agents. For example, antisense oligonucleotides, which are able to inhibit gene expression with exquisite specificity, are often used by those of ordinary skill to elucidate the function of particular genes, for example to distinguish between the functions of various members of a biological pathway. This specific inhibitory effect has, therefore, been harnessed by those skilled in the art for research uses.

The specificity and sensitivity of oligonucleotides is also harnessed by those of skill in the art for therapeutic uses. For example, the following U.S. patents demonstrate palliative, therapeutic and other methods utilizing antisense oligonucleotides. U.S. Pat. No. 5,135,917 provides antisense oligonucleotides that inhibit human interleukin-1 receptor expression. U.S. Pat. No. 5,098,890 is directed to antisense oligonucleotides complementary to the c-myb oncogene and antisense oligonucleotide therapies for certain cancerous conditions. U.S. Pat. No. 5,087,617 provides methods for treating cancer patients with antisense oligonucleotides. U.S. Pat. No. 5,166,195 provides oligonucleotide inhibitors of HIV. U.S. Pat. No. 5,004,810 provides oligomers capable of hybridizing to herpes simplex virus Vmw65 mRNA and inhibiting replication. U.S. Pat. No. 5,194,428 provides antisense oligonucleotides having antiviral activity against influenza virus. U.S. Pat. No. 4,806,463 provides antisense oligonucleotides and methods using them to inhibit HTLV-III replication. U.S. Pat. No. 5,286,717 provides oligonucleotides having a complementary base sequence to a portion of an oncogene. U.S. Pat. No. 5,276,019 and U.S. Pat. No. 5,264,423 are directed to phosphorothioate oligonucleotide analogs used to prevent replication of foreign nucleic acids in cells. U.S. Pat. No. 4,689,320 is directed to antisense oligonucleotides as antiviral agents specific to CMV. U.S. Pat. No. 5,098,890 provides oligonucleotides complementary to at least a portion of the mRNA transcript of the human c-myb gene. U.S. Pat. No. 5,242,906 provides antisense oligonucleotides useful in the treatment of latent EBV infections.

It is preferred to target specific genes for antisense attack. “Targeting” an oligonucleotide to the associated nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a foreign nucleic acid from an infectious agent. In the present invention, the target is a cellular gene associated with several immune system disorders and diseases (such as inflammation and autoimmune diseases), as well as with ostensibly “normal” immune reactions (such as a host animal's rejection of transplanted tissue), for which modulation is desired in certain instances. The targeting process also includes determination of a region (or regions) within this gene for the oligonucleotide interaction to occur such that the desired effect, either detection or modulation of expression of the protein, will result. Once the target region have been identified, oligonucleotides are chosen which are sufficiently complementary to the target, i.e., hybridize sufficiently well and with sufficient specificity to give the desired effect.

“Complementary,” as used herein, refers to the capacity for precise pairing between two nucleobases of an oligomeric compound. For example, if a nucleobase at a certain position of an oligonucleotide is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of an antisense compound need not be 100% complementary to that of its target nucleic acid to be specifically hybridizable. An oligonucleotide is specifically hybridizable when binding of the oligonucleotide to the target DNA or RNA molecule interferes with the normal function of the target DNA or RNA to cause a decrease or loss of function, and there is a sufficient degree of complementarity to avoid non-specific binding of the oligonucleotide to non-target sequences under conditions in which specific binding is desired, i.e., under physiological conditions in the case of in vivo assays or therapeutic treatment, or in the case of in vitro assays, under conditions in which the assays are performed.

Moreover, an oligonucleotide may hybridize over one or more segments such that intervening or adjacent segments are not involved in the hybridization event (e.g., a loop structure or hairpin structure). It is preferred that the antisense compounds described herein comprise at least 70%, or at least 75%, or at least 80%, or at least 85% sequence complementarity to a target region within the target nucleic acid, more preferably that they comprise at least 90% sequence complementarity and even more preferably comprise at least 95% or at least 99% sequence complementarity to the target region within the target nucleic acid sequence to which they are targeted. For example, an antisense compound in which 18 of 20 nucleobases of the antisense compound are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, an antisense compound which is 18 nucleobases in length having 4 (four) non-complementary nucleobases which are flanked by two regions of complete complementarity with the target nucleic acid would have 77.8% overall complementarity with the target nucleic acid and would thus fall within the scope of the described embodiments. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656).

Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489). In some preferred embodiments, homology, sequence identity or complementarity, between the oligomeric and target is between about 50% to about 60%. In some embodiments, homology, sequence identity or complementarity, is between about 60% to about 70%. In preferred embodiments, homology, sequence identity or complementarity, is between about 70% and about 80%. In more preferred embodiments, homology, sequence identity or complementarity, is between about 80% and about 90%. In some preferred embodiments, homology, sequence identity or complementarity, is about 90%, about 92%, about 94%, about 95%, about 96%, about 97%, about 98%, about 99% or about 100%.

As used herein, “hybridization” means the pairing of complementary strands of oligomeric compounds. In the embodiments described herein, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, the phrase “stringent hybridization conditions” or “stringent conditions” refers to conditions under which a compound will hybridize to its target sequence, but to a minimal number of other sequences. Stringent conditions are sequence-dependent and will be different in different circumstances and as described herein. “Stringent conditions” under which oligomeric compounds hybridize to a target sequence are determined by the nature and composition of the oligomeric compounds and the assays in which they are being investigated.

### Modifications

As is known in the art, a nucleoside is a base-sugar combination. The base portion of the nucleoside is normally a heterocyclic base sometimes referred to as a “nucleobase” or simply a “base.” The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound; however, linear compounds are generally preferred. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

It known in the art that oligomers made of natural RNA or DNA nucleobases for the purposes of antisense applications can have reduced efficacy in vivo compared to in vitro conditions. This loss of efficacy is a consequence of a host of factors including cell penetrance and structural stability. To circumvent these problems much work has focused on making modifications of the oligomers such that they retain their targeting ability and mechanism of action while remaining structurally stable. The targets of modification include the phosphodiester backbone of the molecule, the sugar ring or the purine or pyrimidine bases structure. Loss of cell penetrance is attributed to the natural charge of the DNA or RNA oligomer at neutral pH preventing passage by diffusion through the lipid bilayer. Modifications to the oligomer components that reduce the net charge are beneficial to cellular penetration. This is often accomplished through, for example, the modification of the phosphodiester backbone of the molecule. The loss of structural stability is attributed to the action of endogenous endonucleases and exonucleases that cleave the phosphodiester bonds internally or from either the 5′ or 3′ ends of the oligomer. This cleavage can render the DNA or RNA oligonucleotide inactive toward its target.

Common modifications of the various components of the oligonucleotide are summarized in Gallo et al., ((2003) “Design and applications of modified oligonucleotides”. Brazilian Journal of Medical and Biological Research 36: 143-151). For instance, phosphoramidate, phosphorothioate, methylphosphonate and N3′ to P5′ phosphoramidate modifications can be incorporated into the backbone of the molecule. On the sugar structure, both hydrogen, fluorine, amine, o-alkyl (such as 2′-O methoxyethyl) and alkyl groups have been incorporated in place of the naturally occurring atoms. The 2′-O methoxyethyl ribofuranosyl nucleotides confer additional nuclease resistance by addition of the modification at the 3′ end, and in another embodiment, addition of the modification at both the 3′ and ‘5 ends of the same molecule. The modifications would leave phosphorothioate sequences to support Ribonuclease H activity. In addition, expanding the pentose sugar ring by one carbon results in a class of hexitol nucleic acids (Kang et al., 2004 “Inhibition of MDR1 gene expression by chimeric HNA antisense oligonucleotides”. Nucleic Acids Research 32: 4411-4419) with improved properties. Another class of modification involves the replacement of the phosphodiester backbone with repetitive N-(2-aminoethyl) glycine to which the purine or pyrimidine bases are attached via a methyl carbonyl linker (reviewed in Pellestor et al., “The use of peptide nucleic acids for in situ identification of human chromosomes”. Journal of Histochemistry & Cytochemistry 53: 395-400).

It is appreciated by one skilled in the art that a combination of these types of modifications is useful in designing molecules with the desired properties. For instance, phosphorothioate backbone modification may be made at the 5′ and/or 3′ end of the oligomer for increased stability against exonucleases while other sugar modifications may be made in the center of the molecule which would be advantageous for other properties such as enhanced targeting, endonuclease protection and mechanism (Uhlmann E, Ryte A, Peyman A, “Studies on the mechanism of stabilization of partially phosphorothioated oligonucleotides against nucleolytic degradation” in Antisense Nucleic Acid Drug dev. 1997, August; 7(4):345-50). Such molecules that consist of different classes of modifications like those described above are often called chimeric molecules or gapmers.

Phosphorothioate oligonucleotides have a recognized record of good clinical safety, but there are as yet no data on chronic administration. Given the severity and lack of disease modifying treatment for Alzheimer's, treatment with phosphorothioate oligonucleotides could be argued to have an excellent risk/benefit profile. For oligomers used to treat younger patients, for example Down's Syndrome patients, any modification of chemistry can be considered valuable if it reduces exposure to modified oligonucleotides in any way. Hence, the shorter sequence even by 5 nucleotides reduces the length of the molecule by >10%, whilst retaining human specific sequences, primarily at the 5′ half of the oligonucleotide.

Disclosed herein are oligonucleotides including the following oligomers: Human OL-1:

(SEQ ID NO: 1)
5′ TGC ACC TTT GTT TGA ACC CAC ATC TTC TGC AAA
GAA CAC CAA 3′
and

Human OL-101, a 37-mer equivalent to OL-1 shortened by 5 nucleotides from the 3′ end:

(SEQ ID NO: 3)
5′ TGC ACC TTT GTT TGA ACC CAC ATC TTC TGC AAA
GAA C 3′.

In certain embodiments, the oligonucleotides provided herein may have phosphorothioate backbone modifications. In further embodiments, either of OL-1 or OL-101 may have phosphorothioate backbone modifications.

In a yet further embodiment is provided OL-120, which is the OL-1 nucleobase sequence with a phosphorothioate backbone for 5 nucleotides at both the 5′ end and at the 3′ end, and with pyrimidines internally having phosphorothioate chemistry. In another yet further embodiment is provided OL-1011, which is the OL-101 nucleobase sequence with a phosphorothioate backbone for 5 nucleotides at both the 5′ end and at the 3′ end, and with pyrimidines internally having phosphorothioate chemistry.

For example, in further embodiments, an oligonucleotide provided herein may have a sequence selected from human

5′ TpsGpsCps ApsCpsC TTT GTT TGA ACC CAC ATC TTC TGC AAA GAA CpsApsC psCpsApsA 3′ and human

5′ TpsGpsCps ApsCpsC TTT GTT TGA ACC CAC ATC TTC TGC A ApsA psGpsApsApsC 3′,

wherein each “ps” represents a phosphorothioate nucleoside linkage, and wherein either of said sequences may have pyrimidines internally having phosphorothioate chemistry.

In addition to the above oligomers, provided herein are the human OL-1 and human OL-101 nucleobase sequences with a phosphorothioate backbone and a total of five 2-O-methoxyethyl-modified ribofuranosyl nucleotides on the 3′ end of the oligonucleotide, and the Human OL-1 and Human OL-101 nucleobase sequences with a phosphorothioate backbone and total of ten 2-O-methoxyethyl-modified ribofuranosyl nucleotides, five on each end of the oligonucleotide.

In addition to the exemplary modifications discussed above, oligonucleotides as disclosed herein may have any of the following modifications discussed in more detail below.

### Modified Internucleoside Linkages

Specific examples of preferred antisense compounds include oligonucleotides containing modified backbones or non-natural-internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriaminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′ 5′ linkages, 2′ 5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

### Modified Sugar and Internucleoside Linkages: Mimetics

In other preferred antisense compounds, e.g., oligonucleotide mimetics, both the sugar and the internucleoside linkage (i.e. the backbone), of the nucleotide units are replaced with novel groups. The nucleobase units are maintained for hybridization with an appropriate target nucleic acid. One such compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497 1500.

Some embodiments are oligonucleotides with phosphorothioate backbones and oligonucleosides with heteroatom backbones, and in particular—CH₂—NH—O—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂—[wherein the native phosphodiester backbone is represented as—O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

### Modified Sugars

Modified antisense compounds may also contain one or more substituted sugar moieties. Preferred are antisense compounds, preferably antisense oligonucleotides, comprising one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly preferred are O[(CH₂)ₙ)]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH2CH2OCH3, also known as 2′-O—(2-methoxyethyl) or 2′-MOE) (Martin et al., Helv. Chim. Acta, 1995, 78, 486 504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′—O—CH₂—O—CH₂—N(CH₃)₂, also described in examples hereinbelow.

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′ 5′ linked oligonucleotides and the 5′ position of 5′ terminal nucleotide. Antisense compounds may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

A further preferred modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is preferably a methylene (—CH₂—)ₙ group bridging the 2′ oxygen atom and the 4′ carbon atom wherein n is 1 or 2. LNAs and preparation thereof are described in WO 98/39352 and WO 99/14226.

### Natural and Modified Nucleobases

Antisense compounds may also include nucleobase (often referred to in the art as heterocyclic base or simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine[1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858 859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289 302, Crooke, S. T. and Lebleu, B., ed., CRC Press, 1993. Certain of these nucleobases are particularly useful for increasing the binding affinity of the compounds described herein. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

### Conjugates

Another modification of the antisense compounds involves chemically linking to the antisense compound one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of the compounds described herein. Representative conjugate groups are disclosed in International Patent Application PCT/US92/09196, filed Oct. 23, 1992, and U.S. Pat. No. 6,287,860, the entire disclosure of which are incorporated herein by reference. Conjugate moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Antisense compounds may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

Another embodiment is to use peptide nucleic acid (PNA) chemistry to stabilize the sequences of OL-1 h, and other antisense oligonucleotides claimed here, to nucleases and to impart other attractive features. PNAs are synthetic DNA analogues in which the phosphodiester backbone is replaced by repetitive units of N-(2-aminoethyl) glycine to which the purine and pyrimidine bases are attached via a methyl carbonyl linker. This unique chemical makeup provides PNA with unique hybridization characteristics. Unlike DNA and RNA, the PNA backbone is not charged. Consequently, there is no electrostatic repulsion when PNA hybridizes to its target nucleic acid sequence, giving a higher stability to the PNA-DNA or PNA-RNA duplexes than the natural homo- or heteroduplexes. This greater stability results in higher thermal melting temperature (Tm) values than are ob-served for DNA-DNA or DNA-RNA duplexes (Jensen et al. 1997). An additional consequence of the polyamide backbone is that PNAs hybridize virtually independently of the salt concentration. Therefore, the Tm of PNA-DNA duplex is barely affected by low ionic strength. This significantly facilitates the hybridization with the PNAs. The unnatural backbone of PNAs also means that PNAs are particularly resistant to protease and nuclease degradation (Demidov et al. 1993). Because of this resistance to the enzyme degradation, the lifetime of PNAs is extended both in vivo and in vitro. PNAs hybridize to cDNA or RNA in a sequence dependent manner, according to the Watson-Crick hydrogen bonding scheme. In contrast to DNA, PNA can bind in either parallel or anti-parallel fashion and can hybridize with either single-stranded or double-stranded DNA. Homopyrimidine PNAs, as well as PNAs containing a high proportion of pyrimidine residues, bind to cDNA sequences to form highly stable (PNA) 2-DNA triplex helixes displaying high Tm. In these triplexes, one PNA strand hybridizes to DNA through standard Watson-Crick base-pairing rules, while the other PNA strand binds to DNA through Hoogsteen hydrogen bonds. The resulting structure is called P-loops (Nielsen 2001). In addition, PNA-DNA hybridization is significantly more affected by base mismatches than DNA-DNA hybridization. A single mismatch in a mixed PNA-DNA 15-mer duplex decreases the Tm by up to 15° C., whereas in the corresponding DNA-DNA complex, a single mismatch decreases the Tm by only 11° C. (Giesen et al. 1998). This high level of discrimination at the single-base level has indicated that short PNA probes could offer high specificity and has thus allowed the further development of several efficient PNA-based strategies for molecular investigations and diagnosis.

Since its introduction, an increasing number of applications of PNA technology have been described, confirming the high potential of peptide nucleic acids as efficient tools for molecular biology investigations.

PNA molecules were first used in antigene and antisense assays. Several in vitro studies demonstrated the ability of PNAs to inhibit both eukaryotic translation and transcription (Hanvey et al. 1992; Vickers et al. 1995; Boffa et al. 1996).

### Chimeric Compounds

It is not necessary for all positions in a given compound to be uniformly modified, and in fact more than one of the aforementioned modifications may be incorporated in a single compound or even at a single nucleoside within an oligonucleotide.

Other embodiments also include antisense compounds which are chimeric compounds. “Chimeric” antisense compounds or “chimeras” are antisense compounds, particularly oligonucleotides, which contain two or more chemically distinct regions, each made up of at least one monomer unit, i.e., a nucleotide in the case of an oligonucleotide compound. Chimeric antisense oligonucleotides are thus a form of antisense compound. These oligonucleotides typically contain at least one region wherein the oligonucleotide is modified so as to confer upon the oligonucleotide increased resistance to nuclease degradation, increased cellular uptake, increased stability and/or increased binding affinity for the target nucleic acid. An additional region of the oligonucleotide may serve as a substrate for enzymes capable of cleaving RNA:DNA or RNA:RNA hybrids. By way of example, RNAse H is a cellular endonuclease which cleaves the RNA strand of an RNA:DNA duplex. Activation of RNase H, therefore, results in cleavage of the RNA target, thereby greatly enhancing the efficiency of oligonucleotide-mediated inhibition of gene expression. The cleavage of RNA:RNA hybrids can, in like fashion, be accomplished through the actions of endoribonucleases, such as RNAseL which cleaves both cellular and viral RNA. Cleavage of the RNA target can be routinely detected by gel electrophoresis and, if necessary, associated nucleic acid hybridization techniques known in the art.

Chimeric antisense compounds may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

The compounds described herein may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds encompass any pharmaceutically acceptable salts, esters, or salts of such esters, or any other compound which, upon administration to an animal, including a human, is capable of providing (directly or indirectly) the biologically active metabolite or residue thereof.

The term “pharmaceutically acceptable salts” refers to physiologically and pharmaceutically acceptable salts of the compounds described herein: i.e., salts that retain the desired biological activity of the parent compound and do not impart undesired toxicological effects thereto. For oligonucleotides, preferred examples of pharmaceutically acceptable salts and their uses are further described in U.S. Pat. No. 6,287,860, which is incorporated herein in its entirety.

The formulation may include EDTA or its salts as a preservative in a range of 1 nM to 50 mM with pH adjusted to between pH 7 and pH 9, in the presence or absence of other compounds and salts. The EDTA is added to reduce oxidation of phosphorothioate oligonucleotide moieties, and more specifically to reduce de-purination of the biologically active phosphorothioate oligonucleotide. The EDTA will be contained in the most preferred formulation of water for injection containing sodium chloride and sodium and potassium phosphates as commonly referred to as phosphate buffered saline.

The oligonucleotides of the present invention can be utilized as therapeutic compounds, diagnostic tools and as research reagents and kits. The term “therapeutic uses” is intended to encompass prophylactic, palliative and curative uses wherein the oligonucleotides of the invention are contacted with animal cells either in vivo or ex vivo. As used herein, “animal” includes but is not limited to a human.

The antisense compounds can be utilized for diagnostics and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics, the compounds described herein, either alone or in combination with other compounds or therapeutics, can be used as tools in differential and/or combinatorial analyses to elucidate expression patterns of a portion or the entire complement of genes expressed within cells and tissues.

As one non-limiting example, expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17 24; Celis, et al., FEBS Lett., 2000, 480, 2 16), SAGE (serial analysis of gene expression) (Madden, et al., Drug Discov. Today, 2000, 5, 415 425), READS (restriction enzyme amplification of digested cDNAs) (Prashar and Weissman, Methods Enzymol., 1999, 303, 258 72), TOGA (total gene expression analysis) (Sutcliffe, et al., Proc. Natl. Acad. Sci. U.S.A., 2000, 97, 1976 81), protein arrays and proteomics (Celis, et al., FEBS Lett., 2000, 480, 2 16; Jungblut, et al., Electrophoresis, 1999, 20, 2100 10), expressed sequence tag (EST) sequencing (Celis, et al., FEBS Lett., 2000, 480, 2 16; Larsson, et al., J. Biotechnol., 2000, 80, 143 57), subtractive RNA fingerprinting (SuRF) (Fuchs, et al., Anal. Biochem., 2000, 286, 91 98; Larson, et al., Cytometry, 2000, 41, 203 208), subtractive cloning, differential display (DD) (Jurecic and Belmont, Curr. Opin. Microbiol., 2000, 3, 316 21), comparative genomic hybridization (Carulli, et al., J. Cell Biochem. Suppl., 1998, 31, 286 96), FISH (fluorescent in situ hybridization) techniques (Going and Gusterson, Eur. J. Cancer, 1999, 35, 1895 904) and mass spectrometry methods (To, Comb. Chem. High Throughput Screen, 2000, 3, 235 41).

The antisense compounds described herein are useful for research and diagnostics, because these compounds hybridize to nucleic acids encoding a portion of APP. For example, oligonucleotides that are shown to hybridize with such efficiency and under such conditions as disclosed herein as to be effective APP inhibitors will also be effective primers or probes under conditions favoring gene amplification or detection, respectively. These primers and probes are useful in methods requiring the specific detection of nucleic acid molecules encoding APP and in the amplification of said nucleic acid molecules for detection or for use in further studies of APP. Hybridization of the antisense oligonucleotides, particularly the primers and probes described herein, with a nucleic acid encoding at least a portion of APP can be detected by means known in the art. Such means may include conjugation of an enzyme to the oligonucleotide, radiolabelling of the oligonucleotide or any other suitable detection means. Kits using such detection means for detecting the level of either a portion of APP or APP in its entirety in a sample may also be prepared.

The oligonucleotides of the present invention can be further used to detect the presence of APP-specific nucleic acids in a cell or tissue sample. For example, radiolabeled oligonucleotides can be prepared by ³²P labeling at the 5′ end with polynucleotide kinase (Sambrook et al., Molecular Cloning. A Laboratory Manual, Cold Spring Harbor Laboratory Press, 1989, Volume 2, pg. 10.59). Radiolabeled oligonucleotides are then contacted with cell or tissue samples suspected of containing APP mRNAs (and thus APP proteins), and the samples are washed to remove unbound oligonucleotide. Radioactivity remaining in the sample indicates the presence of bound oligonucleotide, which in turn indicates the presence of nucleic acids complementary to the oligonucleotide, and can be quantitated using a scintillation counter or other routine means. Expression of nucleic acids encoding these proteins is thus detected.

Radiolabeled oligonucleotides of the present invention can also be used to perform autoradiography of tissues to determine the localization, distribution and quantitation of APP's, their cleavage products, or other APP derivatives, for research, diagnostic or therapeutic purposes. In such studies, tissue sections are treated with radiolabeled oligonucleotide and washed as described above, then exposed to photographic emulsion according to routine autoradiography procedures. The emulsion, when developed, yields an image of silver grains over the regions expressing an APP gene. Quantitation of the silver grains permits detection of the expression of mRNA molecules encoding these proteins and permits targeting of oligonucleotides to these areas.

Analogous assays for fluorescent detection of expression of APP nucleic acids can be developed using oligonucleotides of the present invention which are conjugated with fluorescein or other fluorescent tags instead of radiolabeling. Such conjugations are routinely accomplished during solid phase synthesis using fluorescently-labeled amidites or controlled pore glass (CPG) columns. Fluorescein-labeled amidites, CPG are available from, e.g. Glen Research, Sterling Va.

The antisense compounds can also be applied in the areas of drug discovery and target validation. The use of the compounds and preferred target segments identified herein in drug discovery efforts to elucidate relationships that exist between APP and a disease state, phenotype, or condition is also contemplated. These methods include detecting or modulating APP comprising contacting a sample, tissue, cell, or organism with the compounds described herein, measuring the nucleic acid or protein level of APP or a portion thereof, and/or a related phenotypic or chemical endpoint at some time after treatment, and optionally comparing the measured value to a non-treated sample or sample treated with a further compound. These methods can also be performed in parallel or in combination with other experiments to determine the function of unknown genes for the process of target validation or to determine the validity of a particular gene product as a target for treatment or prevention of a particular disease, condition, or phenotype.

The specificity and sensitivity of antisense is also harnessed by those of skill in the art for therapeutic uses. Antisense compounds have been employed as therapeutic moieties in the treatment of disease states in animals, including humans. Antisense oligonucleotide drugs, including ribozymes, have been safely and effectively administered to humans and numerous clinical trials are presently underway. It is thus established that antisense compounds can be useful therapeutic modalities that can be configured to be useful in treatment regimes for the treatment of cells, tissues and animals, especially humans.

For therapeutic uses, an animal suspected of having a disease or disorder which can be treated or prevented by modulating the expression or activity of at least a portion of the APP is, for example, treated by administering oligonucleotides in accordance with this invention. The oligonucleotides of the invention can be utilized in pharmaceutical compositions by adding an effective amount of an oligonucleotide to a suitable pharmaceutically acceptable diluent or carrier. Workers in the field have identified antisense, triplex and other oligonucleotide compositions which are capable of modulating expression of genes implicated in viral, fungal and metabolic diseases. Antisense oligonucleotides have been safely administered to humans and several clinical trials are presently underway. It is thus established that oligonucleotides can be useful therapeutic instrumentalities that can be configured to be useful in treatment regimes for treatment of cells, tissues and animals, especially humans.

For therapeutics, an animal, preferably a human, suspected of having a disease or disorder which can be treated by modulating the expression of at least a portion of an APP, including the AβP cleavage product of APP, is treated by administering antisense compounds described herein. For example, in one non-limiting embodiment, the methods comprise the step of administering to the animal in need of treatment, a therapeutically effective amount of an APP inhibitor. The APP inhibitors effectively inhibit the expression of the APP or the APP. In one embodiment, the activity or expression of APP in an animal is inhibited by about 10%. Preferably, the activity or expression of APP in an animal is inhibited by about 30% to about 70%. Thus, the oligomeric antisense compounds modulate expression of APP mRNA by at least 10%, by at least 15%, by at least 20%, by at least 25%, by at least 30%, by at least 35%, by at least 40%, by at least 45%, or by at least 70%.

The antisense compounds described herein may also be utilized in pharmaceutical compositions, both therapeutically and prophylactically. The pharmaceutical formulations, which may conveniently be presented in unit dosage form, may be prepared according to conventional techniques well known in the pharmaceutical industry (see, for example, Remington's Pharmaceutical Sciences). Such techniques include the step of bringing into association the active ingredients with one or more pharmaceutical carrier(s) or excipient(s). The carrier(s) must be “acceptable” in the sense of being compatible with the other ingredients of the formulation and not deleterious to the recipient thereof.

The formulation and dosing of therapeutic compositions and their subsequent administration is believed to be within the skill of those in the art. In general, for therapeutics, a patient in need of such therapy is administered an oligonucleotide in accordance with the invention, commonly in a pharmaceutically acceptable carrier, in doses ranging from 0.01 μg to 100 g per kg of body weight depending on the age of the patient and the severity of the disorder or disease state being treated. In certain embodiments, the dose may be 20 mg/week. Further, the treatment regimen may last for a period of time which will vary depending upon the nature of the particular disease or disorder, its severity and the overall condition of the patient, and may extend from being continuously administered, to administration several times daily, to once every 20 years.

Following treatment, the patient is monitored for changes in his/her condition and for alleviation of the symptoms of the disorder or disease state. The dosage of the oligonucleotide may either be increased in the event the patient does not respond significantly to current dosage levels, or the dose may be decreased if an alleviation of the symptoms of the disorder or disease state is observed, or if the disorder or disease state has been ablated. In any case, the optimal dosage is therapeutically effective for the disease being treated. The phrase “therapeutically effective” is intended to qualify the amount of active ingredients used in the treatment of a disease or disorder. This amount will achieve the goal of reducing or eliminating the disease or disorder.

Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years. In the case of in individual known or suspected of being prone to a disease state due to the accumulation of AβP, such as Alzheimer's disease or Down's Syndrome, prophylactic effects may be achieved by administration of preventative doses, ranging from 0.01 μg to 100 g per kg of body weight, once or more daily, to once every 20 years. In like fashion, an individual may be made less susceptible to the impairment of cognitive function due to the accumulation of AβP's in portions of the brain.

The pharmaceutical compositions of the present invention may be administered in a number of ways depending upon convenience, patient compliance, and/or desired efficacy of the administration route. The route may be oral, parenteral (including subcutaneous, intraperitoneal, intradermal, intramuscular, intravenous, and intraarticular, any of these by injection or infusion), intracranial, intrathecal, intramedullary, and intraventricular, any of these by injection or infusion), intranasal, pulmonary, (including by inhalation or insufflation of powders or aerosol or by nebulizer), transmucosal, transdermal, rectal or topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Compositions for parenteral, intrathecal or intraventricular administration include aqueous and non-aqueous (oily) sterile injection solutions, emulsions, foams and liposome-containing formulations of the active compounds which may contain sterile aqueous solutions which may also contain buffers, diluents and other suitable additives, such as, but not limited to, penetration enhancers, carrier compounds, aqueous and non-aqueous sterile suspensions which may include suspending agents and thickening agents, and other pharmaceutically acceptable carriers or excipients. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Suitable lipophilic solvents or vehicles include fatty oils such as sesame oil, or synthetic fatty acid esters, such as ethyl oleate or triglycerides, or liposomes.

Emulsions are typically heterogenous systems of one liquid dispersed in another in the form of droplets usually exceeding 0.1 μm in diameter. Emulsions may contain additional components in addition to the dispersed phases, and the active drug which may be present as a solution in either the aqueous phase, oily phase or itself as a separate phase. Microemulsions are also contemplated. Emulsions and their uses are well known in the art and are further described in U.S. Pat. No. 6,287,860, which is incorporated herein in its entirety.

Formulations include liposomal formulations. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The pharmaceutical formulations and compositions may also include surfactants. The use of surfactants in drug products, formulations and in emulsions is well known in the art. Surfactants and their uses are further described in U.S. Pat. No. 6,287,860, which is incorporated herein in its entirety.

In one embodiment, various penetration enhancers are employed to effect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

The antisense compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The antisense compounds may also be formulated in rectal compositions such as suppositories or retention enemas, e.g., containing conventional suppository bases such as cocoa butter, polyethylene glycol, or other glycerides.

Antisense compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 μg to 100 g per kg of body weight, and may be given once or more daily, weekly, monthly or yearly, or even once every 2 to 20 years.

One of skill in the art will recognize that formulations are routinely designed according to their intended use, i.e. route of administration.

In one related embodiment, therapeutic treatment of an animal may include treatment of a patient with an oligonucleotide of the invention in conjunction with other therapeutic modalities in order to increase the efficacy of a treatment regimen. Other therapeutic modalities will be well known to those skilled in the art of treating disease states due to accumulation of AβP in the brain, such as AD or DS, and may include other therapies designed to alleviate or ameliorate the decreased cognitive function associated with these diseases. In the context of the invention, the term “treatment regimen” is meant to encompass therapeutic, palliative, and prophylactic modalities.

In another related embodiment, the compositions may contain one or more antisense compounds, particularly oligonucleotides, targeted to a first nucleic acid and one or more additional antisense compounds targeted to a second nucleic acid target. Alternatively, compositions may contain two or more antisense compounds targeted to different regions of the same nucleic acid target. Numerous examples of antisense compounds are known in the art. Two or more combined compounds may be used together or sequentially.

The following examples illustrate the invention and are not intended to limit the same. Those skilled in the art will recognize, or be able to ascertain through routine experimentation, numerous equivalents to the specific substances and procedures described herein. Such equivalents are considered to be within the scope of the present invention.

The invention is further illustrated by the following examples.

### Example 1

### Synthesis and Purification of Oligonucleotides

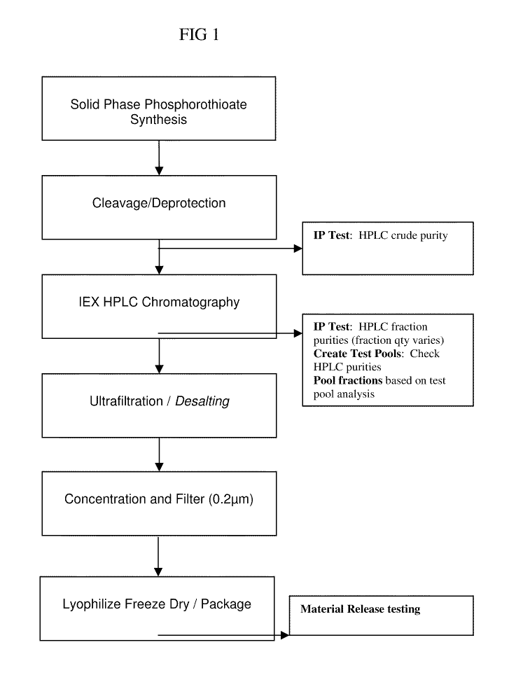

Oligonucleotides may be made by methods well known in the art. Additionally, they may be synthesized and purified as shown in the flow diagram of FIG. 1, a method by which OL-1 was made. Such a procedure should yield a substance with the following characteristics:
Appearance—white powder, visually free from contaminants;
Purity as identified via HPLC=97%; and pH 1% w/v solution=7.8.

### Example 2

### Murine model of Down's Syndrome

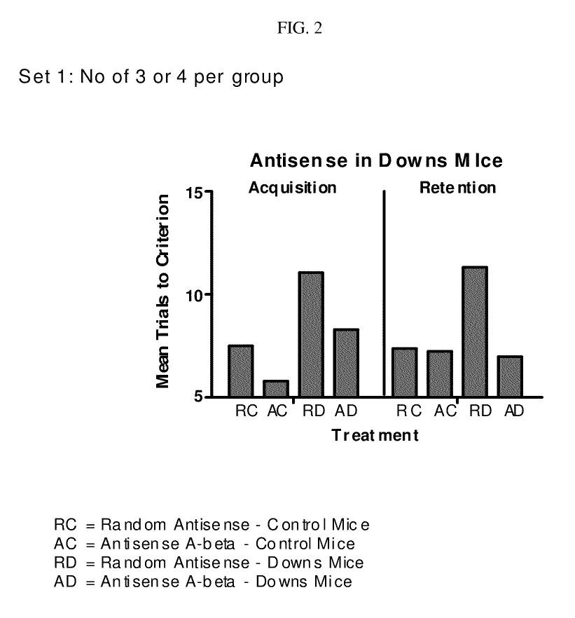

This example illustrates the efficacy of antisense oligonucleotides according to the present invention to improve acquisition and retention in the segmental trisomic 16 (Ts65Dn) mice. Ts65Dn mice have been engineered with a triplicated portion of mouse chromosome 16, which is syntenic to the distal end of human chromosome 21. This strain can be a murine model for human Down's Syndrome, a genetic disorder characterized by the triplication of at least a portion of chromosome 21, and learning and memory deficits, similar to those observed in Alzheimer's disease. Ts65Dn mice display phenotypic abnormalities which resemble those seen in Down's Syndrome, and have been shown to have short- and long-term spatial memory defects. (Demas et al., Behav. Brain Res., 1996, 82, 85-92). See FIG. 2.

Several similarities between Alzheimer's disease and some physiological manifestations of aging in Ts65Dn mice have been reported (Salehi et al, Neuron, 2006, 51, 29-42). One of the manifestations is the increased gene dose and expression of APP in Ts65Dn mice, due to the segmental trisomy of mouse chromosome 16, which includes a third copy of the murine App gene, the mouse ortholog of human APP, the gene encoding APP. Mutations in APP have been associated with increased expression of APP and with familiar Alzheimer's disease (FAD) (Goates et al, Nature, 1991, 349, 704-706). Thus, it can be inferred that over-expression of APP could be one of the causes for the manifestation and loss of memory and acquisition in these mice.

Increased App gene dose in Ts65Dn mice has been shown to cause increased levels of App mRNA and APP (Salehi et al, Neuron 51: 29-42 (2006)). Because the over-expression of the App gene has been associated with an increase in beta-amyloid plaques in the brain, which in turn has been associated with the cognitive defects seen in Alzheimer's disease, it has been postulated that antisense inhibition of the App gene product should have a favorable effect in preventing, or even reversing cognitive impairment.

Murine OL-1 or a random oligonucleotide was delivered by intravenous (IV) administration to control (AC) or Down's (AD) mice, and behavioral testing was conducted to determine how many attempts or how much time mice required to learn or to remember a task, which was reports as Mean Trials to Criterion.” Such testing used has been demonstrated to require areas of the brain known to be involved in learning, memory, and the pathology of AD. Results are shown in FIG. 2.

### Example 3

### Determination of Serum Vs. Brain Concentration of OL-1h

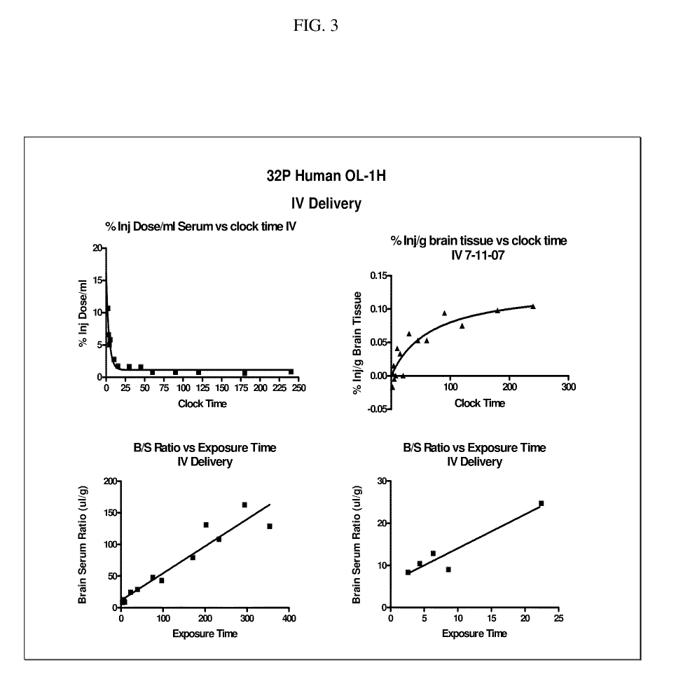

Radiolabeled OL-1 h was delivered intravenously (IV) to mice and concentration in brain versus serum measured in minutes. In FIG. 3 below, the first panel shows that when radioactively labeled OL-1 was injected into the mouse IV, then the OL-1 within an hour had cleared from the blood circulation to reach a lower concentration in blood. The second upper right panel demonstrates that OL-1 has entered the brain with a time course that reaches a plateau between 100 and 300 minutes post injection. The lower 2 panels illustrate that OL-1 in the brain steadily increases compared to the amount in the blood, shown on 2 different timescales. These graphs present the data in the upper panels again but as a ratio of B or brain/S or serum.

### Example 4

### Delivery of Human OL-1h into Mice Via Subcutaneous (subQ) Delivery

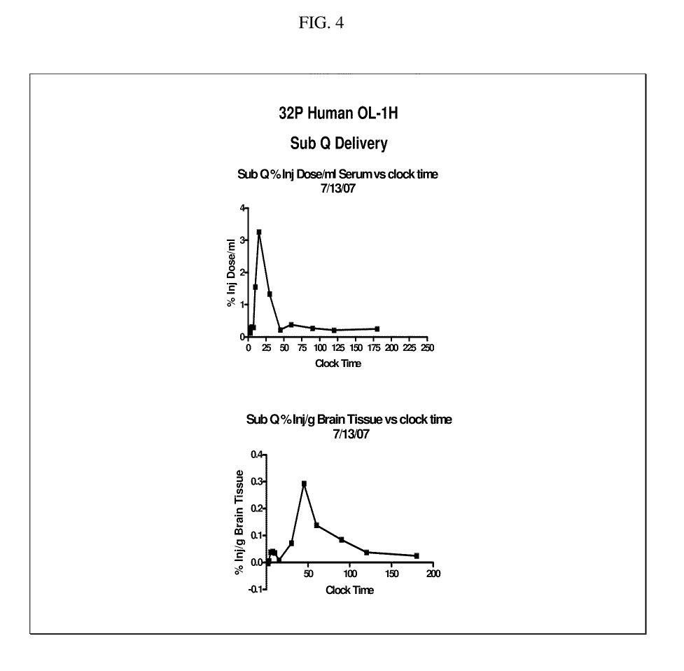

Radiolabeled OL-1 h was delivered subcutaneously (subQ) to mice and concentration in brain versus serum measured in minutes. In FIG. 4 below, the first panel shows that OL-1 within an hour has entered the general circulation, then reached a peak, then declined. In the bottom panel, OL-1 has crossed the blood brain barrier, entering the brain from the general circulation. The small amount of OL-1 remaining in the brain is anticipated to be sufficient to block production of Amyloid Precursor Protein. Subcutaneous injection is a practical alternative to IV injection, and more amenable to treatment outside the doctor's office.

### Example 5

### Oral Delivery of Human OL-1h into Mice

Mice were administered 1,000,000 cpm of the human antisense labeled with ³²P by oral administration. At various times (10-480 min), arterial serum was taken from the carotid artery and the brain harvested. The blood was centrifuged at 4000×g for 10 min and 50 microliter of serum removed and counted in a gamma counter. The pineal and pituitary were removed, the brain washed of excess blood and meninges removed, and the brain weighed and counted in a gamma counter. The results were expressed as the cpm present in one milliliter (ml) or gram (g) of the blood or brain, respectively, divided by the cpm administered orally and multiplied by 100. This yielded the percent of the administered dose which was present in one ml or g of serum or brain. The results are the combined results of n=4/time point conducted on 3 separate days with 2 separate radioactive labelings. The results show that radioactivity was present in blood and brain. Table 1 shows the % cpm in the serum at various time points post administration.

TABLE 1
PerCent of Dose in Serum Post Oral Administration

| | |
|---|---|
| Clock | |
| Time | |
| (min) | % CPM |

| | | | | |
|---|---|---|---|---|
| 2.0 | 0.032 | | 0.005 | 0.000 |
| 3.0 | 0.008 | 0.008 | 0.028 | 0.006 |
| 4.0 | 0.133 | 0.007 | 0.109 | 0.000 |
| 5.0 | 0.592 | | 0.019 | 0.022 |
| 7.5 | 0.165 | 0.391 | 0.031 | 0.000 |
| 10.0 | 0.021 | | 0.011 | 0.000 |
| 15.0 | 0.013 | 0.014 | 0.304 | 0.000 |
| 20.0 | 0.059 | | 0.020 | 0.298 |
| 30.0 | 0.011 | 0.301 | 0.106 | 0.046 |
| 45.0 | 0.040 | 0.158 | 0.036 | 0.201 |
| 60.0 | 0.045 | 0.012 | 0.066 | 0.074 |
| 90.0 | 0.045 | 0.112 | 0.032 | 0.078 |
| 120.0 | 0.080 | 0.021 | 0.085 | 0.059 |
| 180.0 | 0.260 | 0.233 | 0.382 | 0.264 |
| 240.0 | 0.983 | 0.335 | 0.276 | 0.255 |
| 360.0 | | 0.196 | 0.267 | 0.264 |
| 480.0 | | 0.195 | 0.331 | 0.119 |

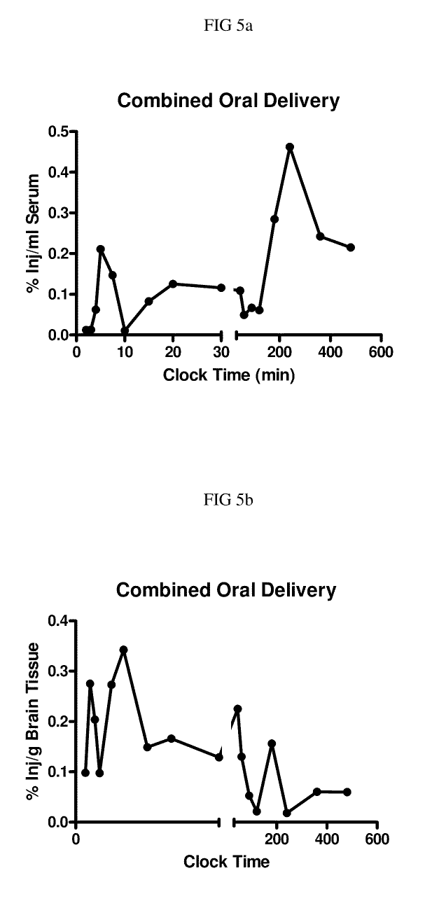

The amount of radioactivity expressed as the percent of starting oral administration in the brain is tabulated in Table 2. Levels in brain were similar to those found after intravenous administration, although blood levels were somewhat lower. The average cpm in serum or brain vs. time are shown in FIGS. 5a and 5b, respectively. Radioactive levels were not corrected for any degradation that might have occurred during GI absorption.

TABLE 2
PerCent of Dose in Brain Post Oral Administration

| | |
|---|---|
| Clock | |
| Time | |
| (min) | % CPM |

| | | | | |
|---|---|---|---|---|
| 2.0 | 0.051 | | 0.145 | |
| 3.0 | 0.356 | 0.203 | 0.384 | 0.158 |
| 4.0 | 0.094 | 0.505 | 0.012 | |
| 5.0 | 0.194 | | 0.080 | 0.018 |
| 7.5 | 0.727 | 0.070 | 0.022 | |
| 10.0 | 0.647 | | 0.038 | |
| 15.0 | 0.191 | 0.216 | 0.040 | |
| 20.0 | 0.383 | | 0.075 | 0.040 |
| 30.0 | 0.126 | 0.170 | 0.077 | 0.142 |
| 45.0 | 0.518 | 0.087 | 0.275 | 0.020 |
| 60.0 | 0.062 | 0.223 | 0.188 | 0.047 |
| 90.0 | 0.012 | 0.089 | 0.063 | 0.047 |
| 120.0 | 0.037 | 0.010 | 0.029 | 0.010 |
| 180.0 | 0.511 | 0.046 | 0.040 | 0.027 |
| 240.0 | 0.020 | 0.014 | 0.020 | 0.018 |
| 360.0 | | 0.055 | 0.092 | 0.034 |
| 480.0 | | 0.103 | 0.060 | 0.016 |

### Example 6

### Intranasal Administration of Antisense Oligonucleotides and Distribution in Brain Regions

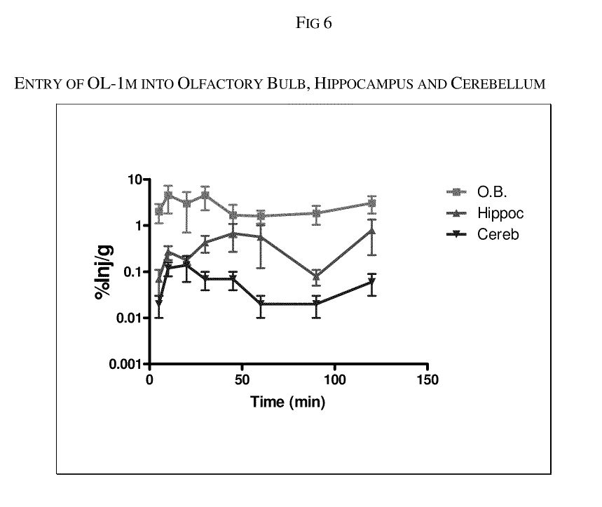

Two microliter of ³²P radioactively labeled OL-1m in physiological saline was deposited at the base of the cribriform plate of adult CD-1 mice. At designated time points, the brains were dissected and the percentage of starting cpm was determined as previously described. Each time point has an n=6 mice. The results shown in FIG. 6 (ordinate on a log scale) demonstrate that OL-1m enters and persists in each of three areas of the brain after intranasal administration. The three areas were chosen because of their involvement in the pathology of AD, and because previous data had demonstrated that OL-1m enters and persists in these areas. Brain areas examined: Cerebellum, Hippocampus, and Olfactory Bulb.

### Example 7

### Intranasal (in) Administration of OL-1m Restores Learning (Acquisition) and Memory (Retention)

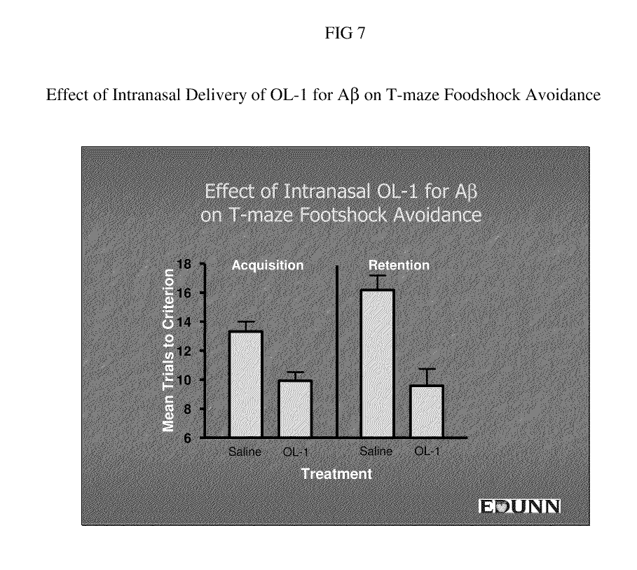

Intranasal administration of OL-1m (6 ug) 3 times at two week intervals improves acquisition and retention of T-maze footshock avoidance in 12 month old SAMP8 mice compared to the saline treated controls (N=6 per group) when tested two weeks after the third administration. The results are shown in FIG. 7.

### Example 8

### Comparison of Antisense Oligonucleotides Levels in Serum and Brain after IV or Intranasal Administration

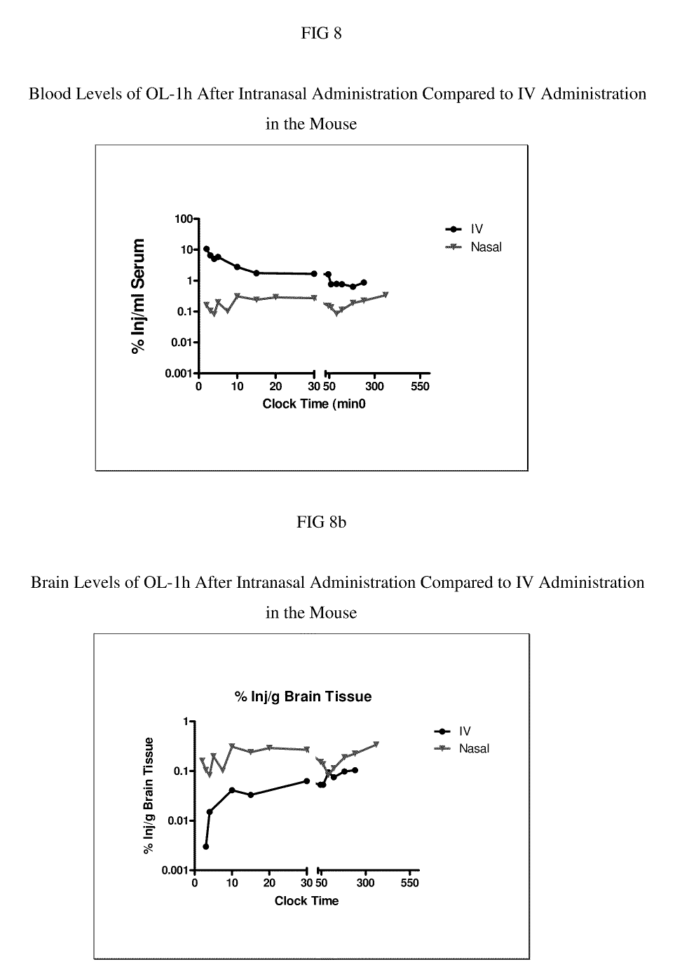

Two microliters of ³²P radioactively labeled OL-1 h in physiological saline were deposited at the base of the cribriform plate of adult CD-1 mice, or diluted in saline and administered IV. Each time point has an n=2 mice for intranasal administration and n=1 for IV. At various time points the % of the original input cpm found in the serum and brain was determined. The results are shown in FIG. 8.

FIG. 8a shows that the blood levels of OL-1 h after intranasal administration are approximately 100 times less than the blood levels achieved by IV administration; please note the log scale of the ordinate.

FIG. 8b shows that when the entry of intranasal-dosed OL-1 h into the brain was compared to the entry into brain after IV dosing, the results were not similar to the blood level data of FIG. 8a. OL-1 h demonstrated efficient entry into the brain after intranasal delivery and reached a plateau after 10 minutes, whereas IV delivered OL-1 h in the same period of time had entered the brain but had reached only 10% of the level achieved by the intranasal administration. The IV delivered OL-1 h was reaching a plateau at 50 minutes, at which time the OL-1 h appeared to have approximated the same level as the intranasal dose.

In addition to the Examples outlined above, it is expected that an experiment in which OL-1 were delivered by intramuscular (IM) injection would yield similar results.

From the foregoing description, one skilled in the art can easily ascertain the essential characteristics of this invention, and without departing from the spirit and scope thereof, can make various changes and modifications of the invention to adapt it to various usages and conditions.

1. A nucleic acid molecule comprising a nucleotide sequence that is at least 90% identical to SEQ ID NO: 1.

2. The nucleic acid of claim 1, wherein the nucleotide sequence is at least 95% identical to SEQ ID NO: 1.

3. A nucleic acid molecule comprising the nucleotide sequence of SEQ ID NO: 1.

4. The nucleic acid of claim 1, wherein the nucleotide sequence is complementary to a polynucleotide that encodes the amino acid sequence of SEQ ID NO: 2, and wherein the nucleic acid inhibits the expression of an amyloid beta protein (AβP) portion of an amyloid precursor protein.

5. The nucleic acid of claim 1, wherein the nucleic acid is an antisense oligonucleotide.

6. The nucleic acid of claim 1, wherein the nucleic acid is 10 to 80 nucleobases in length.

7. The nucleic acid of claim 1, wherein the nucleic acid is 35 to 45 nucleobases in length.

8. The nucleic acid of claim 1, wherein the nucleic acid is 37, 38, 39, 40, 41, 42, or 43 nucleobases in length.

9. The nucleic acid of claim 3, wherein the nucleic acid is 42 nucleobases in length.

10. The nucleic acid of claim 1, wherein the nucleic acid comprises at least one modified internucleoside linkage.

11. The nucleic acid of claim 10, wherein the modified internucleoside linkage is a phosphorothioate linkage.

12. The nucleic acid of claim 9, wherein the nucleic acid comprises at least one phosphorothioate linkage.

13. The nucleic acid of claim 1, wherein the nucleic acid comprises at least one modified sugar moiety.

14. The nucleic acid of claim 13, wherein the modified sugar moiety is a 2′-O-methoxyethyl sugar moiety.

15. The nucleic acid of claim 1, wherein the nucleic acid comprises at least one modified nucleobase.

16. The nucleic acid of claim 15, wherein the modified nucleobase is a 5-methylcytosine.

17. The nucleic acid of claim 1, wherein the nucleic acid is a chimeric oligonucleotide.

18. The nucleic acid of claim 1, wherein the nucleic acid is capable of crossing the blood-brain barrier.

19. A composition comprising the nucleic acid of claim 1 and a pharmaceutically acceptable excipient, carrier, or diluent.

20. The composition of claim 19, further comprising a colloidal dispersion system.

21. The composition of claim 19, wherein the nucleic acid is an antisense oligonucleotide.

22. The composition of claim 19, wherein said composition may be administered via oral, parenteral, subcutaneous, intraperitoneal, intradermal, intramuscular, intravenous, intraarticular, intracranial, intrathecal, intramedullary, intraventricular, intranasal, pulmonary, transmucosal, transdermal, rectal, topical, dermal, buccal, sublingual or intraocular administration.

23. The composition of claim 19, wherein said composition may be administered intravenously.

24. A method of inhibiting the expression of amyloid precursor protein in cells or tissues comprising administering the nucleic acid of claim 1 to the cells or tissues so that expression of the amyloid precursor protein is inhibited.

25. A method of inhibiting the expression of amyloid precursor protein in cells or tissues comprising administering the composition of claim 19, to the cells or tissues so that expression of the amyloid precursor protein is inhibited.

26. A method of treating a subject having a disease or condition associated with amyloid beta protein precursor or amyloid beta protein, comprising:
administering a therapeutically or prophylactically effective amount of the nucleic acid of claim 1 to said subject so that expression of amyloid precursor protein is inhibited.

27. A method of treating a subject having a disease or condition associated with amyloid beta protein precursor or amyloid beta protein, comprising:
administering a therapeutically or prophylactically effective amount of the composition of claim 19, to said subject so that expression of amyloid precursor protein is inhibited.

28. The method of claim 26, wherein the disease or condition is one in which amyloid metabolism is inhibited.

29. The method of claim 27, wherein the disease or condition is one in which amyloid metabolism is inhibited.

30. The method of claim 26, wherein the disease or condition is any variant of Alzheimer's disease, mild cognitive dysfunction, Down's Syndrome, Parkinson's disease, familial Alzheimer's disease, homozygosity for the apolipoprotein E4 allele, Dementia pugilistica (including head trauma), Hereditary Cerebral Hemorrhage with amyloidosis of the Dutch type (HCHWA-D), or vascular dementia with amyloid angiopathy.

31. The method of claim 27, wherein the disease or condition is any variant of Alzheimer's disease, mild cognitive dysfunction, Down's Syndrome, Parkinson's disease, familial Alzheimer's disease, homozygosity for the apolipoprotein E4 allele, Dementia pugilistica (including head trauma), Hereditary Cerebral Hemorrhage with amyloidosis of the Dutch type (HCHWA-D), or vascular dementia with amyloid angiopathy.

32. A method of prophylactic treatment of a patient not previously diagnosed with AD, Parkinson's Disease, MCI, dementia, or pre-dementia, which patient displays an elevated level of AβP in the brain, comprising the step of:
administering to said patient an amount of the nucleic acid of claim 1 effective to prevent or delay development of AD, Parkinson's Disease, MCI, dementia, or pre-dementia.

33. A method of prophylactic treatment of a patient not previously diagnosed with AD, Parkinson's Disease, MCI, dementia, or pre-dementia, which patient displays an elevated level of AβP in the brain, comprising the step of:
administering to said patient an amount of the composition of claim 19, effective to prevent or delay development of AD, Parkinson's Disease, MCI, dementia, or pre-dementia.

34. A method of enhancing cognitive function comprising the step of:
administering to a subject an amount of the nucleic acid of claim 1 effective to enhance cognitive function.

35. A method of enhancing cognitive function comprising the step of:
administering to a subject an amount of the composition of claim 19 effective to enhance cognitive function.

36. The method of claim 26, wherein said administration is oral, parenteral, subcutaneous, intraperitoneal, intradermal, intramuscular, intravenous, intraarticular, intracranial, intrathecal, intramedullary, intraventricular, intranasal, pulmonary, transmucosal, transdermal, rectal, topical, dermal, buccal, sublingual or intraocular.

37. The method of claim 27, wherein said administration is oral, parenteral, subcutaneous, intraperitoneal, intradermal, intramuscular, intravenous, intraarticular, intracranial, intrathecal, intramedullary, intraventricular, intranasal, pulmonary, transmucosal, transdermal, rectal, topical, dermal, buccal, sublingual or intraocular.

38. The method of claim 26, wherein said administration is intravenous.

39. The method of claim 27, wherein said administration is intravenous.

40. A nucleic acid molecule comprising (i) the nucleotide sequence of SEQ ID NO: 1; or (ii) a nucleotide sequence complementary to a polynucleotide that encodes the amino acid sequence of SEQ ID NO: 2,
wherein the nucleic acid is 42 nucleobases in length and wherein the nucleic acid comprises phosphorothioate linkages.
